(19) United States
(12) Patent Application Publication
(10) Pub. No.: US 2008/0074618 A1
(43) Pub. Date: Mar. 27, 2008

(54) Fatigue detection device using encoded light signals

(76) Inventor: Baohua Qi, Columbus, IN (US)

Correspondence Address: Baohua Qi, 6076 Basswood Drive, Columbus, IN, 47201, US

(21) Appl. No.: 11/803,294

(22) Filed: May 14, 2007

Related U.S. Application Data
(60) Provisional application No. 60/800,474, filed on May 15, 2006.

(51) Int. Cl. A61B 3/10

(52) U.S. Cl. 351/221

(57) ABSTRACT

A method and apparatus for detecting fatigue level. According to this method, eyelid movement and eye reflectivity are measured simultaneously by using encoded light signals. Eye reflectivity is used to detect early fatigue while eyelid movement is used for monitoring late fatigue. For more accurately and reliably measuring reflectivity, the emitting light intensity is adjusted according to background light conditions. Devices based on this invention can work during both daytime and nighttime.

This present application claims priority from U.S. provisional application No. 60/800,474 having the same tile as the present invention and filed on May 15, 2006.

### CROSS-REFERENCE TO RELATED APPLICATIONS

1. Ludmirsky A., & Zigler A., Method and apparatus for fatigue detection, U.S. Pat. No. 4,967,186

2. Torch, W. C., Method and apparatus for voluntary communication, U.S. Pat. No. 6,246,344

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

This invention relates to a method and apparatus for measuring fatigue or drowsiness level of an individual, and more particularly, a method and apparatus using encoded light signals to detect eye reflectivity and eyelid movement.

### BACKGROUND OF THE INVENTION

Fatigue and distraction contribute to a significant portion of the accidents occurring on US roads. According to NHTSA (The National Highway Traffic Safety Administration) data, there have been more than 56,000 crashes annually in which driver drowsiness or fatigue was cited by police. However, evaluating the road safety impact of driver fatigue is difficult due to the difficulty in detecting the fatigue. Unlike some other problems such as alcohol and drugs that can be detected by measuring the concentration in breath or body fluid, fatigue is a natural internal state change. Most evidences for fatigue, e.g. eye blink rate change, slow response, and driving manner change, are behavior changes, which are the outcomes of the fatigue but not the fatigue itself. It is difficult to use a single measure to reliably quantifying driver fatigue.

A few technologies have been developed for detecting fatigue. According to the approaches used by these technologies, most of them fall into three categories, i.e. physiological measures, driving performance measures, and eye movement detection. In physiological measures, EEG (Electroencephalography) and EMG (Electromyography) have been studied experimentally as both methods to detect fatigue and tools to evaluate and validate other measures. Both EEG and EMG are able to provide direct evidence of fatigue. However, for applications as on-road fatigue detection devices, they are not realistic options due to the difficulties in reliably contacting the electrodes with human body in a comfortable and undistrubing way.

In driving performance measures, driving behaviors, such as braking and accelerating, lane tracking, and headway tracking, in which the distance between the driver's vehicle and that in front is monitored, are used to evaluate the performance of drivers. When an erratic driving or a risky situation is detected, a warning will be triggered. Some of driver performance monitor technologies have been used in commercial vehicles (e.g. Citroen C4). However, driving performance measures are indirect methods that detect the outcomes of fatigue. Road conditions and lighting have significant effects to the effectiveness of driving performance measures, and difficulties exist in distinguishing normal driving and driving with drowsiness.

The eye movement detection includes gaze tracking, eyelid movement detection, and eyelid reflectivity measurement (U.S. Pat. No. 4,967,186). Among these technologies, an eyelid movement detection approach—PERCLOS (Percent Eye Closure) showed its potential for real-world applications. However, a fundamental problem for gaze tracking and eyelid movement detection is that the physical changes in eyes are not likely to be occurring or hardly detected when a person is lightly fatigued. As a result, the driver could have been through a significant period of high crash risk before eye gazing or eye closure changes can be detected. Eyelid reflectivity measurement could provide a more sensitive measure for fatigue, though problems exist in reliability due to effects of environmental light change.

A variety of sensors, including video cameras, IR (Infrared) sensors, piezoelectric sensors, EEG/EMG/EOG devices, accelerometers, LADAR sensors, and temperature sensors have been used for fatigue detection. Among these sensors, video cameras and IR sensors, which have been used for PERCLOS detection and gaze tracking, are reliable, undisturbing, and user-friendly, and thus see more on-road applications. However, video cameras are not low cost sensors. In addition, to process data in real time, usually it needs a powerful computation device to process the image signals acquired from video cameras. This further increases the cost.

An object of the present invention is to provide a user-friendly, undisturbing, and low-cost fatigue detection apparatus that is able to reliably work in both of daytime and nighttime.

### BRIEF SUMMARY OF THE INVENTION

The invention presents an eye movement and reflectivity measurement method and device that only uses light sensors. In this device, eyelid movement and eye reflectivity will be measured simultaneously. Eye reflectivity is used to detect early fatigue while eyelid movement will be used for monitoring late fatigue. Different from other devices, this device will use encoded light signals that include a special emitter ID code and light intensity information, which can be background light intensity and/or emitting light intensity. Since only signals with an ID code are processed, this device is insensitive to environmental light conditions. Additionally, for more accurately and reliably measuring reflectivity, the emitting light intensity is adjusted according to background light conditions. These improvements in signal detection and signal processing allow the system be used with weak light emitting signals in a noisy environment. The device can be installed on glasses (e.g. photochromic sunglasses) and it can work during both daytime and nighttime. The use of infrared (IR) light is preferred since it generates least distraction to the driver.

The technology used in the present invention combines light reflectivity measurement with serial communication. In addition to fatigue detection, this technology can also find its application in a variety of other applications, such as material surface reflectivity examination, skin surface reflectivity examination, and eyelid communication (U.S. Pat. No. 6,246,344).

### BRIEF DESCRIPTION OF THE DRAWINGS

FIG. 1 illustrates the block diagram of a fatigue detection device that includes an emitter and a receiver;

FIG. 2 depicts the block diagram for a realization example of the emitter in the fatigue detection device;

FIG. 3 shows the block diagram for a realization example of the data processing block in the receiver of the fatigue detection device;

FIG. 4 is a waveform chart for signals in the receiver example;

FIG. 5 is the flowchart of the Code Examination routine in the receiver example;

FIG. 6 is the flowchart of the Eye Close Time Examination routine in the receiver example;

FIG. 7 is the flowchart of the Reflectivity Examination routine in the receiver example;

FIG. 8 is the flowchart of the Data Analysis routine in the receiver example.

### DETAILED DESCRIPTION OF THE INVENTION

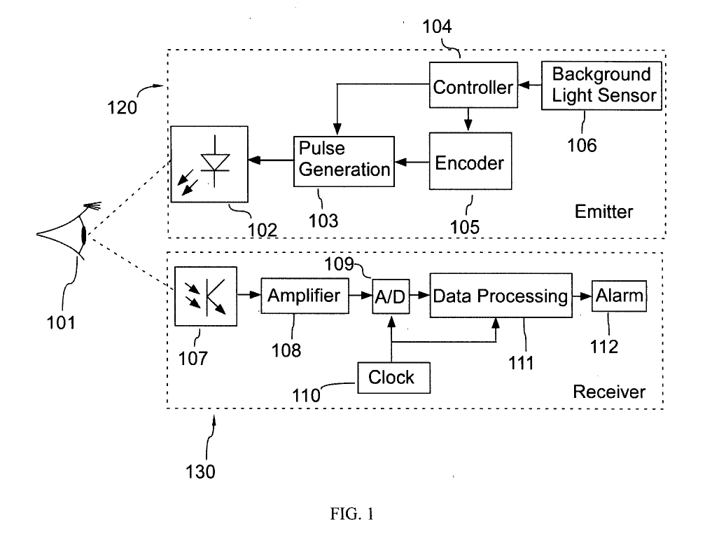

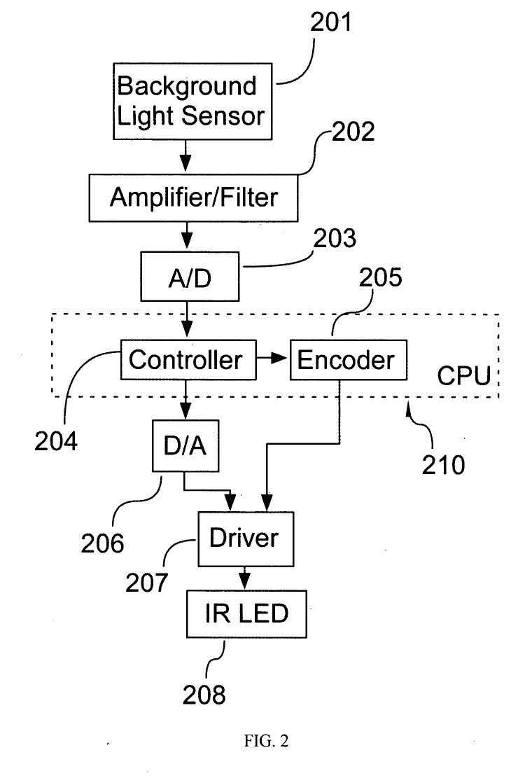

For demonstration purpose, infrared (IR) light is used in the embodiments of the present invention. Referring to FIG. 1, a fatigue detection device includes an emitter 120 and a receiver 130. In the emitter 120, a controller 104 is used to provide an intensity setting for an IR pulse generation circuit 103 according to the sensing values acquired from a background light sensor 106, while an encoder 105 is employed to generate a code including an emitter ID number, emitting light intensity setting, and background light intensity. The IR pulse generation circuit 103 modulates the code generated by the encoder and drives an IR LED 102 with the light intensity setting. The result IR pulses are emitted to a human eye 101 and the reflected signals are obtained by the receiver 130. In the receiver 130, through an amplifier 108, the reflected IR signals acquired from an IR sensor 107 are digitized using an A/D converter 109. The result digital signals are then examined and analyzed in a data processing unit 111. The data processing and A/D conversion are synchronized by a signal generated by a Clock block 110. When a fatigue, i.e. low reflectivity and/or slow eyelid closure, is detected, a warning will be generated by an alarm circuit 112.

A detailed block diagram of the emitter 120 is depicted in FIG. 2. In the emitter, a background light sensor 201 is used to detect the environmental light condition. Signals acquired from the background light sensor 201 are processed through an amplifier/filter 202, and the result signals are converted into digital signals by using an A/D converter 203. A controller 204, which can be realized as a routine running in a CPU 210, calculates the IR emitting intensity based on the environmental light condition and sets the intensity value through a D/A converter 206 to a driver 207 that is used to provide driving signals for an IR LED 208. Then, through an encode 205, which can also be a routine running in the CPU 210, the controller concatenates the background light intensity value and the IR intensity setting value together with an emitter ID code into a pulse sequence. The driver 207 generates control signals based on the intensity setting and the pulse sequence, and the result IR pulses are emitted.

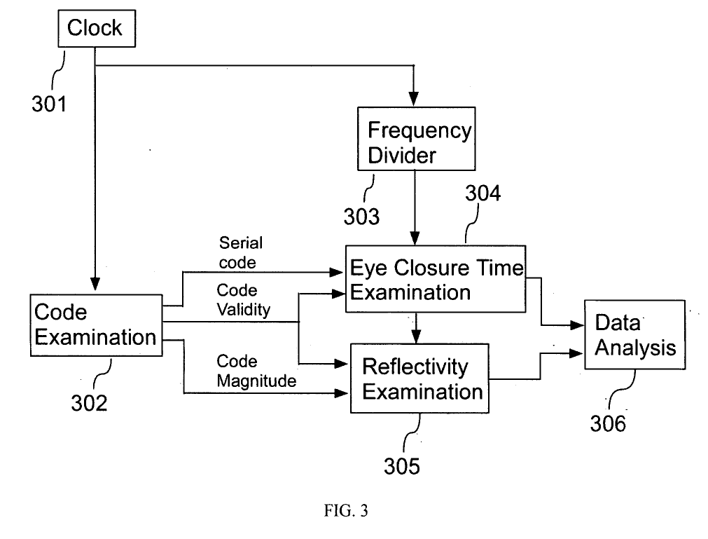

FIG. 3 shows the block diagram for a realization example of the data processing unit 111 (FIG. 1), which includes a Code Examination block 302, an Eye Closure Time Examination block 304, a Reflectivity Examination block 305, and a Data Analysis block 306. The code examination is synchronized by a clock signal 301, while the Eye Closure Time Examination block 304 and the Reflectivity Examination block 305 are synchronized by using a lower frequency signal generated from the Clock signal through a frequency divider 303.

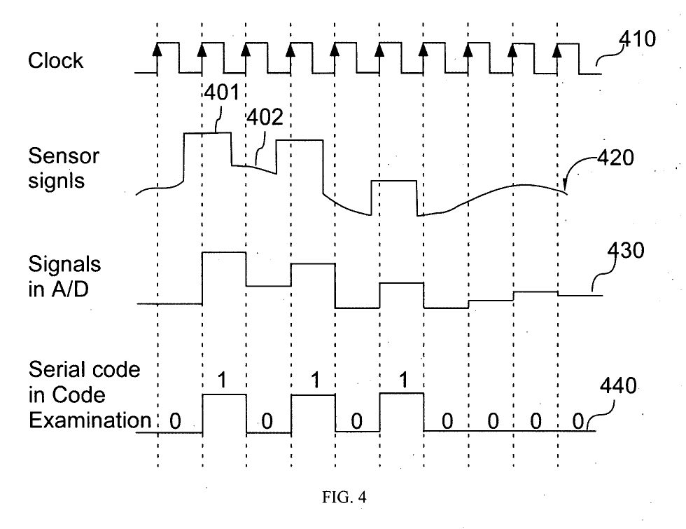

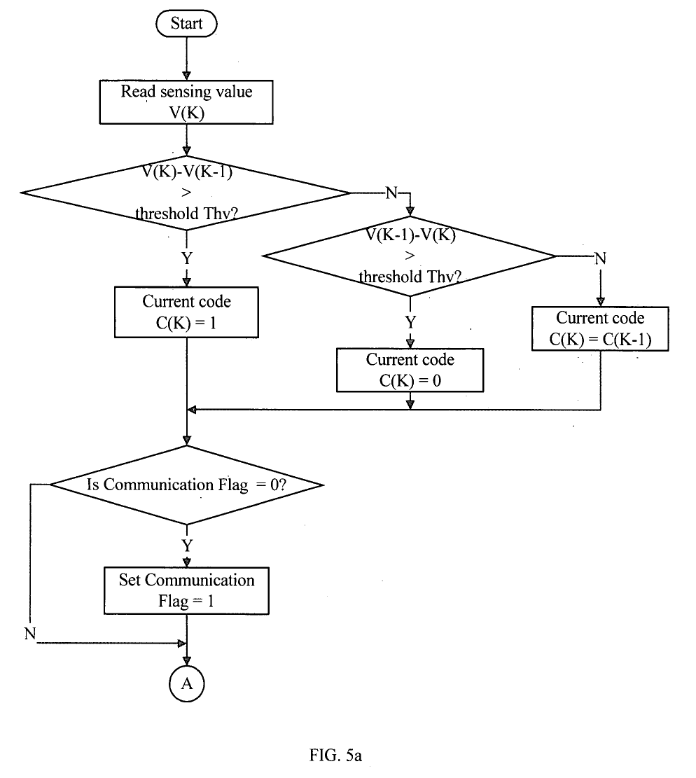

FIG. 4 shows the timing chart of the A/D conversion and code examination. The code examination is realized by using a time interrupt service routine, which is called at every rising edge of a clock signal 410. In the routine, the A/D conversion is triggered and the analog sensing signal 420, which includes a background signal 402 and reflected IR pulses 401, is converted to a digital signal 430. In each sampling period, by examining the digital signal 430, a serial code 440 and its validity flag are generated, and the code magnitude, i.e., the average height of the code pulses is measured. In a device with the emitter and receiver controlled by a single CPU, the serial code may have just the emitter ID code included, since the same CPU controls emitting light intensity based on background light intensity. The CPU can use this information directly in data processing. However, when the emitter and receiver are independent, the serial code needs further include the background light intensity information, and the emitting light intensity information. The code validity flag signal is generated by examining the emitter ID code. If it matches the record in the Code Examination block, the code validity flag is set to 1, otherwise, it is reset to 0.

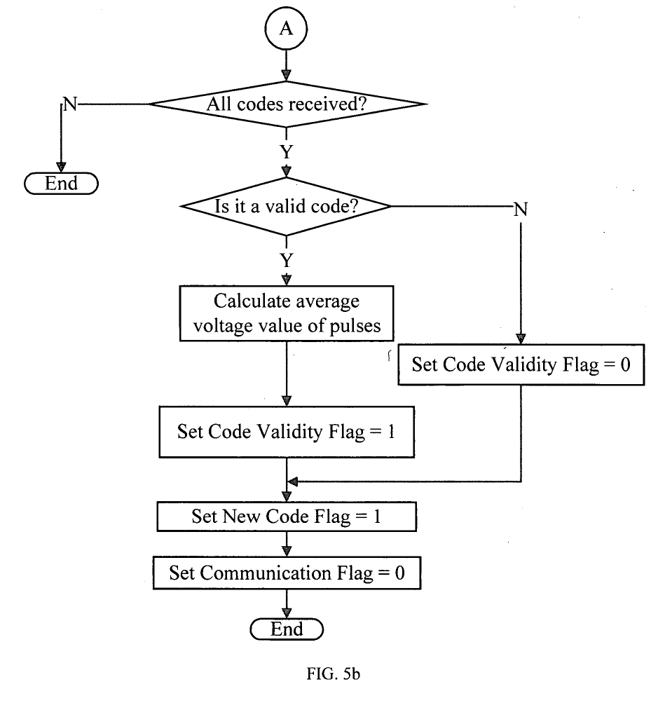

The flowchart for the code examination routine is shown in FIG. 5. After start, the routine triggers an A/D conversion and reads the current digital sensing value V(K) from the A/D converter 109 (FIG. 1), where K is the number of the current sampling period. Then this value is compared with that acquired in the previous sensing period V(K−1). If the difference V(K)−V(K−1) is higher than a threshold Thv, then a rising edge is detected, and the current code value C(K) is set to 1. The code value C(K) will be set to 0 when the value of V(K−1)−V(K) is higher than the threshold Thv (or V(K)′−V(K−1) is lower than −Thv). When the difference between the current sensing value V(K) and the previous value V(K−1) is smaller than the threshold Thv, then the current code value C(K) equals to the previous code value C(K−1). After setting the current code value, the routine checks if the current sampling period is the first one in receiving an IR pulse sequence. If it is, then a Communication Flag is set to 1. After all IR pulses are received, the routine will compare the emitter ID code to its record to examine the validity of the code. If the code matches the record, then a Code Validity Flag is set to 1, and the average pulse height is calculated. The Code Validity Flag is set to 0 when the emitter ID code does not match the record. If all reflected IR signals are received, before the routine ends, the Communication Flag is set to 0 and a New Code Flag is set to 1. The New Code Flag will be cleared to 0 after the information generated by the Code Examination block is processed.

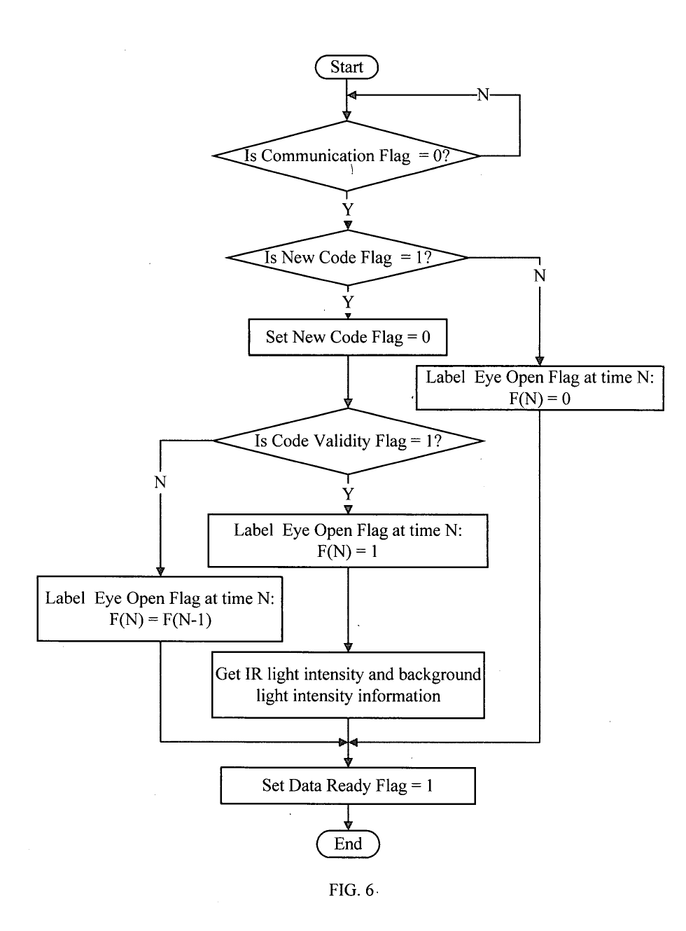

The serial codes generated by the Code Examination Block together with the Code Validity Flag are sent to the Eye Closure Time Examination Block for further processing. The Eye Closure Time Examination Block can also be realized using a time interrupt routine, which has a lower priority than that of the Code Examination Routine, and has an interrupt frequency the same as that for IR emitting. As shown in FIG. 6, after start, the code examination routine checks the status of the Communication Flag. If the Communication Flag is 0, then the New Code Flag is examined. The Eye Open Flag at the current sampling period F(N) is set to 0 when the New Code Flag is 0. If the New Code Flag is 1, the routine sets it to 0 for the communication in next cycle, and checks the validity of the IR code. The value 1 (True) of the Code Validity Flag means a valid IR code is received, upon which, the Eye Open Flag F(N) is set to 1, and the IR light intensity and background light intensity information is calculated from the serial code generated by the Code Examination block (for devices with independent emitter and receiver). When an invalid IR code is received, the Eye Open Flag in the current sampling period will be set to the same value as that in the previous sampling period, i.e., F(N)=F(N−1). Before the routine ends, a Data Ready Flag is set to 1 for data processing in the Reflectivity Examination block.

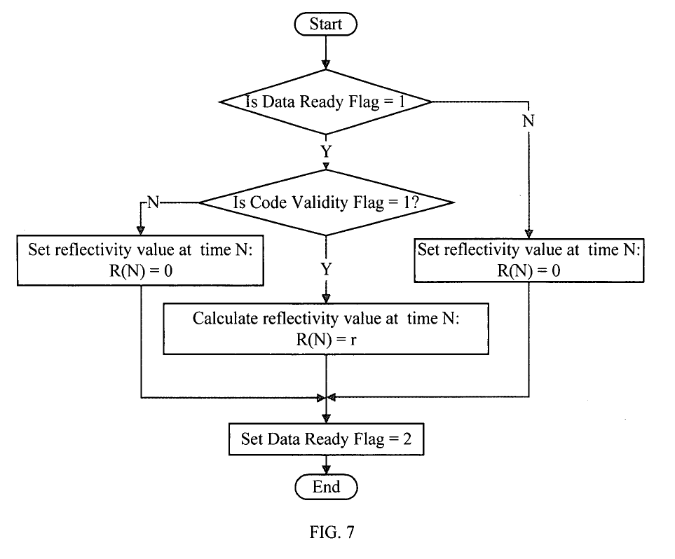

The Reflectivity Examination block 305 (FIG. 3) can be either a separate routine, or incorporated in the Eye Closure Time Examination routine. In a separate routine, referring to FIG. 7, after start, the routine examines the Data Ready Flag generated by the Eye Closure Time Examination routine. If the flag is 1, then the data validity is further examined by checking the status of the Data Validity Flag. The average voltage value of the reflected IR pulses r is associated to R(N), which is the reflectivity value at sampling period N, when the Data Validity Flag is 1. Before the routine ends, the Data Ready Flag is set to 2 for further data analysis.

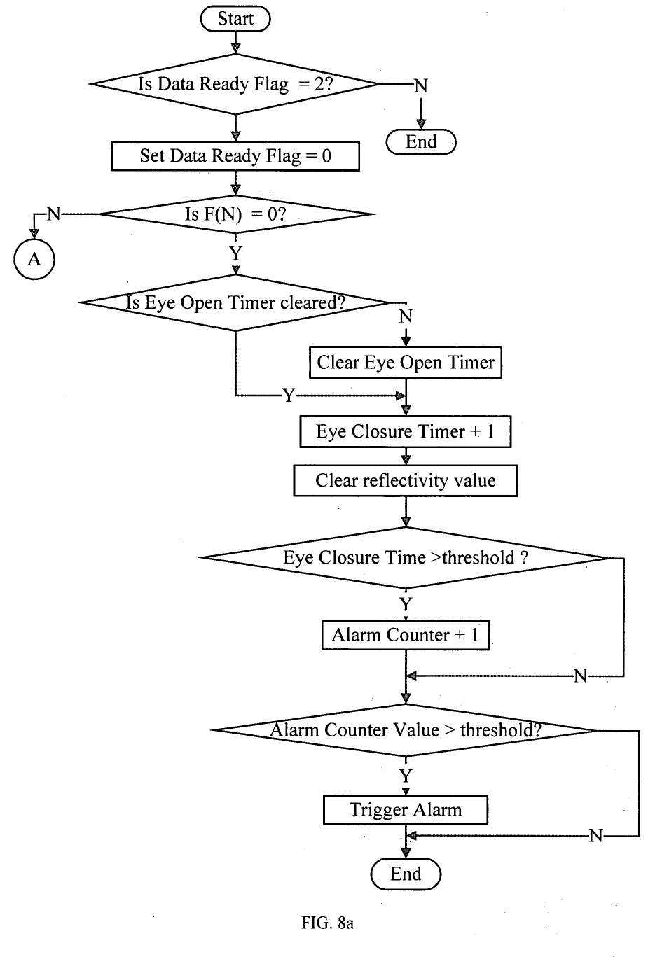

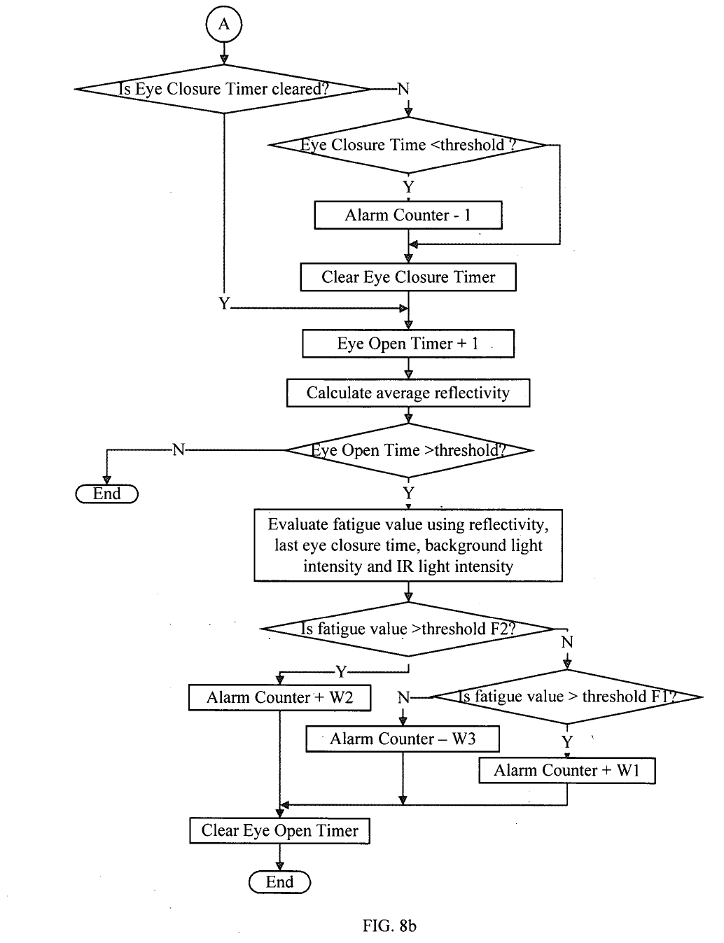

The Eye Open Flag value F(N) and reflectivity value R(N) are sent to a Data Analysis block 306 (FIG. 3) for further processing. As depicted in FIG. 8, in the Data Analysis routine, after start, the Data Ready Flag is examined. If the Data Ready Flag is 2, i.e., the IR reflectivity is calculated, then this flag is set to 0 for calculation in the next cycle, and the Eye Close Flag F(N) is examined, otherwise, the Data Analysis routine ends. When an eye close event is detected, i.e. F(N)=0, an Eye Open Timer is cleared, and average reflectivity value is set to 0. An Eye Closure Timer increments. If the Eye Closure Timer value is higher than a threshold, then an Alarm Counter increments. An alarm will be triggered when the alarm counter value is higher than a threshold.

When an eye open event is detected, i.e., F(N)=1, the value of the eye closure time will be examined and then cleared. If the Eye Closure timer value is less than a threshold, the alarm counter decrements. After the examination for eye closure time, an Eye Open Timer increments and the reflectivity value in the current sampling period is averaged with that in previous sampling periods. If the eye open time is higher than a threshold, a fatigue value is calculated based on the reflectivity, the last eye closure time, background light intensity, and IR light intensity, otherwise, the routine ends. The fatigue value is further compared with two thresholds. When the fatigue value is higher than a threshold F2, the Alarm Counter value increase by W2. A value of W1 will be added to the Alarm Counter if the fatigue value is lower than the threshold F2 but higher than a lower threshold F1. If a fatigue value lower than the threshold F1 is obtained, the Alarm Counter value will be decreased by W3. The Eye Open Timer is cleared when the routine ends, so that the fatigue evaluation using IR reflectivity can only be performed periodically with a rate set by using the eye open time threshold. (IR reflectivity is evaluated only when the eye open time is longer than a threshold)

1. An apparatus for detecting fatigue, said apparatus comprising:
a light emitter for generating light pulses that include an ID (identification) code;
a receiver for receiving and processing reflected light signals that is generated by said light emitter and reflected by human eyes;
in said receiver, said ID code obtained from said reflected light signals is used to calculate eye open time and eye closure time, while the pulse height of said reflected light signals is used for calculating reflectivity of eyes.

2. The apparatus according to claim 1, wherein said light is infrared light.

3. The apparatus according to claim 1, wherein said light emitter further includes a background light sensor.

4. The apparatus according to claim 3, wherein the height of emitting light pulses is adjusted according to background light intensity measured by said background light sensor.

5. The apparatus according to claim 4, wherein said reflectivity is calculated based on said reflected light signals and said height of emitting light pulses.

6. The apparatus according to claim 1, wherein said reflectivity of eyes is used for detecting early fatigue.

7. The apparatus according to claim 1, wherein said eye closure time is used for detecting late fatigue.

8. The apparatus according to claim 1, wherein said eye closure time is calculated by using the time between two received ID codes.

9. An apparatus for detecting fatigue, said apparatus comprising:
a light emitter for generating light pulses that include an ID code and an information code;
a receiver for receiving and processing reflected light signals that is generated by said light emitter and reflected by human eyes;
in said receiver, said ID code obtained from said reflected light signals is used to calculate eye open time and eye closure time, while the pulse height of said reflected light signals is used for calculating reflectivity of eyes.

10. The apparatus according to claim 9, wherein said light is infrared light.

11. The apparatus according to claim 9, wherein said information code includes emitting light intensity information.

12. The apparatus according to claim 9, wherein said light emitter further includes a background light sensor.

13. The apparatus according to claim 12, wherein said information code include background light intensity information.

14. The apparatus according to claim 12, wherein the height of emitting light pulses is adjusted according to background light intensity measured by said background light sensor.

15. The apparatus according to claim 14, wherein said reflectivity is calculated based on said reflected light signals and said height of emitting light pulses.

16. The apparatus according to claim 9, wherein said information code include emitting time information.

17. The apparatus according to claim 16, wherein said eye closure time is calculated by using said emitting time information included in said information code.
